(19) United States
(12) Patent Application Publication
(10) Pub. No.: US 2011/0189669 A1
(43) Pub. Date: Aug. 4, 2011

(54) METHODS OF AIDING IN THE DIAGNOSIS OF PROSTATE CANCER

(75) Inventors: Laki Buluwela, London (GB); Jonathan Waxman, London (GB); Simak Ali, London (GB); Sarah Ngan, London (GB)

(73) Assignee: IMPERIAL INNOVATIONS LIMITED, London (GB)

(21) Appl. No.: 13/000,079

(22) PCT Filed: Jun. 19, 2009
(86) PCT No.: PCT/GB2009/001556

§ 371 Date: Apr. 20, 2011

(30) Foreign Application Priority Data
Jun. 20, 2008 (GB) 0811360.7

(51) Int. Cl. C12Q 1/68; C12Q 1/48

(52) U.S. Cl. 435/6.11; 435/6.14; 435/15; 435/6.18

(57) ABSTRACT

The current invention provides a method for aiding in the assessment of prostate cancer (including metastatic prostate cancer) and/or benign prostate hyperplasia in a patient, wherein the method comprises the step of determining the level of Glycine N-methyltransferase (GNMT) nucleic acid and/or protein in a sample from the patient. The invention also provides compounds that target Glycine N-methyltransferase (GNMT) protein and/or nucleic acid for use in treating prostate cancer. Also provided are screening methods for selecting a compound considered to be useful in treating prostate cancer, comprising the steps of determining the ability of a test compound to reduce GNMT activity and selecting a compound that reduces GNMT activity. The invention also provides methods for aiding in the diagnosis of prostate cancer in a patient comprising obtaining a sample from the patient and assessing said sample for a marker of GNMT activity.

### BACKGROUND

The human prostate is a prototypical androgen-dependent organ. Androgens are required not only for its normal development and growth, but also for structural and functional integrity. The prostate is also a major site for androgen-related pathology. Benign prostatic hyperplasia is the most common proliferative disorder of any internal organ and prostate cancer is the most commonly diagnosed cancer in Western males and the second leading cause of male cancer death.

The growth and development of prostate cancer is initially androgen-dependent and treatment is directed at inhibiting cancer growth by suppression of androgen action or production. Standard treatment involves androgen ablation therapies, mediated either surgically by bilateral orchidectomy, or pharmacologically by the action of anti-androgens (1, 2).

Androgen action is mediated by the androgen receptor (AR). The androgen-bound AR stimulates prostate cancer growth through activation of a transcriptional programme, which facilitates cancer cell proliferation and survival. We have identified the Glycine N-methyltransferase (GNMT) gene as showing a 16-fold increase in expression within 24 hours of androgen treatment. Further analysis has confirmed that GNMT gene expression is stimulated by androgen within 4 hours of treatment and that androgen treatment leads to induction of GNMT protein expression. This demonstrates for the first time that GNMT is an androgen regulated gene in prostate cancer cells. Importantly, GNMT expression has been described by other workers to show a very restricted tissue distribution, primarily being found in the liver, pancreas and prostate (Chen et al. (2000) Genomics 66: 43-47; http://genecards.ccbb.re.kr/cgi-bin/carddisp.pl?gene=GNMT&hom_acc=all).

GNMT is a methyltransferase that methylates glycine to generate sarcosine, the methyl group being donated by S-adenosyl-methionine (SAM), which in turn is converted to S-adenosyl-homocysteine (SAH). We have found that SAH potentiates androgen regulated growth in LNCaP cells. SAH has been reported to be inhibitory for methyltransferase activity but GNMT activity is relatively insensitive to SAH (13) (and therefore likely to have differing sensitivities to compounds than other methyltransferases). By raising intracellular pools of SAH, GNMT potentially functions to regulate protein and DNA methyltransferases, and consequently protein and DNA methylation. Hence GNMT may regulate many cellular processes, including gene expression. Structural information on GNMT is available for human, rat, mouse, rabbit and pig GNMT (20). This data indicates that GNMT is a tetrameric protein that binds 5-methyl-tetrahydrofolate pentaglutamate. Other methyltransferases have been considered as potential drug targets for conditions other than prostate cancer. Consequently, inhibitors for Methyltransferases are being sought (existing inhibitors for methyltransferases include 5-Aza-Cytidine). Folic acid is an allosteric inhibitor of GNMT (Luka et al. (2007) JBC 282: 4069-4075).

Humans with mutations in the GNMT gene have been described in the literature. These individuals had abnormal methionine metabolites, persistent hypermethioninaemia and mild liver disease, but were otherwise normal. Mouse models have been developed where the GNMT gene has been knocked out (Luka et al. (2006) Transgenic Res. 15: 393-397). Homozygous ΔGNMT mice had a complete absence of GNMT protein and its activity in their livers but they did not display any apparent phenotype. However, ΔGNMT mice do exhibit a 7-fold increase in free methionine, a 35-fold increase in SAM and a 3-fold decrease in SAH over wild-type. The Ratio of SAM to SAH increased from 3 to 300 in the livers of homozygous ΔGNMT mice. There is no reported elevation in SAM and methionine in prostate cancer, as might be expected if GNMT were a tumour suppressor gene as argued in the existing literature.

US 2006/0024285 teaches the use of SAM-dependent-methyltransferases (GNMT included) in a method for the prevention or treatment of cancer (including prostate cancer). This method uses GNMT, for example, for the detoxification of a carcinogen, benzo(a)pyrene. There is no suggestion that inhibition of GNMT or other SAM-dependent-methyltransferases would be beneficial for the treatment of cancer.

U.S. Pat. No. 5,994,093 teaches a method of detecting an abnormality of cells comprising comparing the level of GNMT with cells not having the abnormality, the abnormal cells having a decreased level of GNMT. This method is directed to detecting abnormalities in hepatocellular carcinoma cells. The inventors compared the expression of certain genes in normal hepatic cells and tumourous hepatic cells. It was found that fragments of GNMT were expressed at much lower levels or not at all in tumourous hepatic cells. U.S. Pat. No. 5,994,093 in fact teaches that the introduction of GNMT into malignant cells reduced their tumourigenicity. Thus there is no suggestion that the inhibition of GNMT would be beneficial in the treatment of cancer.

US 2004/0101913 also describes the down-regulation of GNMT in hepatocarcinoma cells in comparison with normal liver cells. US 2004/0101913 teaches methods for diagnosing a disease characterised by GNMT expression by providing a biological sample from a patient having clinical symptoms associated with hepatoma and contacting the sample with selected monoclonal antibodies and detecting binding, the presence of such binding indicates the presence of the disease. Diseases that are contemplated include hepatoma and prostate cancer. The malignancies are characterised by down-regulation of expression or inappropriate expression of GNMT. There is no suggestion that inhibition of GNMT would be beneficial in the treatment of cancer.

Collectively, the data presented herein highlight GNMT as a potentially new and important target molecule for prostate cancer. Additionally, the current invention provides for the measurement of the ratio of SAM to SAH and/or sarcosine levels in biological fluids, which may provide quantitative data on disease activity, and new biomarkers to monitor patients' response to treatment for prostate cancer. The current invention also provides biomarkers to assess effects of new treatments for prostate cancer. Also, the measurement of GNMT levels allows investigations into the role of GNMT in regulating the growth of prostate cancer cells.

### SUMMARY OF THE INVENTION

A first aspect of the invention provides a method for aiding in the assessment of prostate cancer (including metastatic prostate cancer) and/or benign prostate hyperplasia in a patient, wherein the method comprises the step of determining the level of GNMT nucleic acid and/or protein in a sample from the patient. The GNMT may be measured in conjunction with Prostate Specific Antigen (PSA) levels in an embodiment of this aspect of the invention. Combining measuring GNMT and PSA levels may provide a more robust and/or definitive assessment of disease state.

The patient may be at risk of developing prostate cancer, for example on the basis of age but may not yet have shown clinical signs of the disease. For example, men older than about 60 years may be at greater risk of prostate cancer than men below the age of 35. Alternatively, the patient may already have been diagnosed with prostate cancer and may or may not have begun treatment for prostate cancer. Additionally, the patient may have already undergone treatment for prostate cancer. The patient who has already undergone treatment for prostate cancer may still have signs of the disease or they may have gone into ‘remission’. It is envisaged that the method of the current invention will be useful in the assessment of, for example, prostate cancer at all the above mentioned ‘stages’ of the disease. It is also envisaged that the compounds of later aspects of the invention will be active against all ‘stages’ of prostate cancer. Further to the explanation provided above, the prostate cancer disease course may be divided into distinct stages. For example, stage II is where the cancer involves more tissue within the prostate but is organ confined, stage III is where the cancer has spread outside the prostate to nearby tissue and stage 1V is where the cancer has metastasised to lymph node or other tissues.

It is preferred that the sample of all aspects of the invention is a urine sample but the sample may also be, for example, a prostate massage urine sample, a blood sample, a blood serum sample, a blood plasma sample, a lymph sample, a sample of seminal fluid or any other sample of body fluid where GNMT secretion may be a sign of prostate cancer. The GNMT protein or nucleic acid may be contained within cells in these samples or it may be extracellular. The GNMT may also be measured in biopsies of suspected cancers. The measurement of mRNA levels in cells in the blood, urine or seminal fluid may provide indications of synthesis of GNMT. Alternatively, detection of GNMT protein by immunohistochemistry and mRNA by in situ hybridisation and real time RT-PCR from prostate biopsied material (or, for example enriched prostate cells, or cells identified as prostate cells, for example as discussed below) may also be useful. Using urine samples and/or samples of seminal fluid may be more convenient and may also be particularly informative, as the urine and/or seminal fluid may accurately reflect prostate conditions.

It is preferred that if blood, seminal fluid, lymphatic circulation or urine is the source of the said sample containing nucleic acid derived from the patient that the sample is enriched for prostate-derived tissue or cells. Enrichment for prostate cells may be achieved using, for example, cell sorting methods such as fluorescent activated cell sorting (FACS) using a prostate-selective antibody such as one directed to prostate-specific membrane antigen (PSMA) (Silver et al., (1997) Clinical Cancer Research 3: 81-85). Alternatively, enrichment may be achieved using magnetic beads or other solid support, for example a column, coated with such a prostate-specific antibody, for example an anti-PSMA antibody. Other examples of antigens that may be suitable in methods of enrichment/purification of prostate cells are epithelial cell surface antigens, which would also facilitate the purification of tumour/epithelial cells from fluids such as blood. Alternatively, cells in the sample may be identified as prostate cells, for example on the basis of prostate-selective antibody/antigens as discussed above without necessarily enriching the cells in the sample. The source of the said sample also includes biopsy material and tumour samples, also including fixed paraffin mounted specimens as well as fresh or frozen tissue.

The method may be used for assessing the likely progression of prostate cancer, metastatic prostate cancer and/or benign hyperplasia in the patient. The method may also be useful for aiding in the diagnosis of, or diagnosing, prostate cancer, metastatic prostate cancer and/or benign hyperplasia in the patient. The method may also or alternatively be useful for aiding in the assessment of the likelihood or likely severity or likely progression of prostate cancer in the patient. This may include assessing the likelihood of the development of complications associated with prostate cancer in the patient, for example arising from metastasis of the prostate cancer. The method may also be useful for assessing prostate function. The method may be useful for distinguishing between benign hyperplasia, prostate cancer and metastatic prostate cancer. This in turn may aid in improving the outcome of these conditions by allowing physicians more accurately to assess these conditions and provide the most appropriate treatments.

The method may be useful for assessing and/or predicting the development of prostate cancer in the patient. GNMT protein or mRNA levels may also be used as a surrogate marker for the development of prostate cancer.

The screening of prostate cancer patients for changes in GNMT protein and/or mRNA levels may be useful for diagnosing those patients that may have or may develop complications associated with prostate cancer.

By “complications associated with prostate cancer” is included such instances as failure to respond to therapy. This may include endocrine therapies (e.g. luteinising hormone-releasing hormone (LHRH) agonists and anti-androgens such as flutamide) and/or chemotherapy. Also, any other unexpected or undesirable outcome in the patient, which is associated with the prostate cancer, may be termed a ‘complication’. Typically, PSA levels are used to monitor the patient's response to treatment and rising PSA levels may be a possible sign of the emergence of resistance to the therapy and/or the re-growth of the tumour.

By “prostate cancer” is included any condition of the cells or tissues of the prostate that has arisen through abnormal cell growth originating from tissues/cells of the prostate. This may include pre-cancerous stages distinguished by abnormal cell growth at one end of the spectrum to metastatic prostate cancer as a more severe form of the disease. Benign hyperplasia may be considered a pre-cancerous stage in some instances.

The response of the patient to treatment for prostate cancer may be assessed using the methods of the current invention. Such treatment may be for prostate cancer or any prostate related disease with which GNMT up-regulation or down-regulation has been associated. Thus, the method may be useful in predicting the future response of the patient to treatment for prostate cancer. The method of the current invention may also be used for assessing the likely progression of response of the patient to treatment for prostate cancer. It may also be useful in prognosis or aiding prognosis.

The normal course of action when deciding whether to treat a patient for prostate cancer, or indeed initially diagnosing the condition, consists of assessing the level and activity of PSA in the patient's urine and assessing the size of the cancer by conducting rectal examinations. This is then followed by a period of “watchful waiting”, when surveillance of PSA levels is carried out (Parekh et al (2007) J. Natl. Cancer Inst. 99: 496-97; Fall et al. (2007) J. Natl. Cancer Inst. 99: 526-532). When changes occur in these parameters physicians are more likely to take action. The reasons for these delays include the potential of subjecting men who would otherwise live healthy lives with indolent cancers to cancer treatments, which are often accompanied by unpleasant side-effects. By assessing the level of GNMT in conjunction with PSA levels, the accuracy of diagnosis and assessment of the need for intervention may be increased. This in turn may lead to a significant improvement in the quality of life of patients. Patients that do not need treatment may be spared the effects of treatment and patients who may not have been considered to be in need of treatment may be provided with life-saving treatments.

The methods of the current invention may also be used for choosing patients for treatment for prostate cancer or for monitoring response of patients to treatment or for monitoring relapse in patients. These treatments may include, but are not limited to, endocrine therapies such as anti-androgens such as flutamide, or other endocrine treatments such as luteinising hormone-releasing hormone (LHRH) agonists. On the basis of such selection the patients may be grouped, for example, into particular patient groups for clinical trials. It is envisaged that clinical trial data may be assessed using the methods of the current invention. The methods may be used, for example, for assessing the progress of patients in clinical trials. The use of GNMT as a biomarker in clinical trials of prostate cancer patients may aid in the assessment of, for example, remission of the cancer.

The methods of the current invention may comprise the steps of (i) obtaining a sample containing nucleic acid and/or protein from the patient; and (ii) determining whether the sample contains a level of GNMT nucleic acid or protein associated with the development, progression or regression (after appropriate treatment) of prostate cancer.

It will be appreciated that determining whether the sample contains a level of GNMT nucleic acid or protein associated with prostate cancer may in itself be diagnostic (or prognostic) of prostate cancer or it may be used by the clinician as an aid in reaching a diagnosis or prognosis.

Thus, measurement of GNMT levels may be performed or considered alongside other measurements or factors, for example, determining the level of PSA, in the sample from the patient and/or measuring the size of any suspect cancer in the patient through digital rectal examination. Any physical examination may also include taking biopsies of suspected cancerous tissue or monitoring other physical indicators of cancer as appropriate. Simultaneous measurement of other hormones or factors may be helpful, such as for example, blood IGF-1 level (Chan et al (1998) Science 279: 563-566). Measurement of GNMT levels may provide more detailed information on the severity of individual disease mechanisms.

It will be appreciated that determination of the level of GNMT in the sample will be useful to the clinician in determining how to manage prostate cancer in the patient. For example, since our research has indicated that elevated levels of GNMT are associated with prostate cancer, the clinician may use the information concerning the levels of GNMT to facilitate decision making regarding treatment of the patient.

The level of GNMT which is indicative of prostate cancer may be defined as the increased level present in samples from patients with prostate cancer relative to levels present in samples from control healthy volunteers. The level of said GNMT protein may be, for example, at least 2 standard deviations higher in a sample from a patient with prostate cancer than the control healthy volunteers. The level of mRNA encoding GNMT may be, for example, at least 2 standard deviations higher in a sample from a patient with prostate cancer.

In any aspect of the invention, the level of GNMT in a sample from the patient may be determined using any suitable protein detection or quantitation method, for example using methods employing antibodies specific for GNMT. Thus, immunoassay techniques, preferably quantitative techniques, may be used, for example an antibody array or captured ELISA technique, as would be understood by a person of skill in the art. Preferred embodiments relating to methods for detecting GNMT protein include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David at al in U.S. Pat. Nos. 4,376,110 and 4,486,530. Other techniques include: Beads-based immunoassay using Luminex type machine; Antibody arrays (including membrane based, or glass based); Proteomic analysis (mass spectrometry, antibody coated biochips using SELDI-TOF technique).

It will be appreciated that other antibody-like molecules may be used in the methods of the invention including, for example, antibody fragments or derivatives which retain their antigen-binding sites, synthetic antibody-like molecules such as single-chain Fv fragments (ScFv) and domain antibodies (dAbs), and other molecules with antibody-like antigen binding motifs.

Bioassays may alternatively be used for measuring GNMT activity, although this may not be preferred, as it may not be convenient to carry out routine assays in this way.

In one preferred embodiment of the invention it is determined whether the level of GNMT nucleic acid, in particular mRNA, is a level associated with prostate cancer. Preferably, the sample contains nucleic acid, such as mRNA, and the level of GNMT is measured by contacting said nucleic acid with a nucleic acid which hybridises selectively to GNMT nucleic acid. This may typically be in the context of a Reverse-Transcriptase Polymerase Chain Reaction (RT-PCR) using at least one primer specific to the GNMT gene encoding GNMT, conducted using standard protocols available in the art. Thus, the PCR primer is an example of a nucleic acid which hybridise selectively to GNMT nucleic acid. RT-PCR may be directed towards regions within the coding region of GNMT or alternatively to the 5′ and/or 3′ untranslated regions, as will be well known to those skilled in the art.

By “selectively hybridising” is meant that the nucleic acid has sufficient nucleotide sequence similarity with the said human nucleic acid that it can hybridise under moderately or highly stringent conditions. As is well known in the art, the stringency of nucleic acid hybridisation depends on factors such as length of nucleic acid over which hybridisation occurs, degree of identity of the hybridizing sequences and on factors such as temperature, ionic strength and GC or AT content of the sequence. Thus, any nucleic acid that is capable of selectively hybridising as said is useful in the practice of the invention.

Nucleic acids which can selectively hybridise to the said human nucleic acid include nucleic acids which have >95% sequence identity, preferably those with >98%, more preferably those with >99% sequence identity, over at least a portion of the nucleic acid with the said human nucleic acid. As is well known, human genes usually contain introns such that, for example, a mRNA or cDNA derived from a gene would not match perfectly along its entire length with the said human genomic DNA but would nevertheless be a nucleic acid capable of selectively hybridising to the said human DNA. Thus, the invention specifically includes nucleic acids which selectively hybridise to GNMT mRNA or cDNA but may not hybridise to a GNMT gene. For example, nucleic acids which span the intron-exon boundaries of the GNMT gene may not be able to selectively hybridise to the GNMT mRNA or cDNA.

Typical moderately or highly stringent hybridisation conditions which lead to selective hybridisation are known in the art, for example those described in Molecular Cloning, a laboratory manual, 2nd edition, Sambrook at al (eds), Cold Spring Harbor Laboratory Press, Cold Spring Harbor, N.Y., USA, incorporated herein by reference. An example of a typical hybridisation solution and protocol is provided in, for example, WO 02/18637.

By “nucleic acid which selectively hybridises” is also included nucleic acids which will amplify DNA from the said GNMT mRNA by any of the well known amplification systems, in particular the polymerase chain reaction (PCR), as noted above.

Although the nucleic acid which is useful in the methods of the invention may be RNA or DNA, DNA is preferred, for example if assessing the patient for GNMT polymorphisms. If assessing expression levels then mRNA may be preferred. Although the nucleic acid that is useful in the methods of the invention may be double-stranded or single-stranded, single-stranded nucleic acid is preferred under some circumstances such as in nucleic acid amplification reactions.

The nucleic acid that is useful in the methods of the invention may be any suitable size. However, for certain diagnostic, probing or amplifying purposes, it is preferred if the nucleic acid has fewer than 10 000, more preferably fewer than 1000, more preferably still from 10 to 100, and in further preference from 15 to 30 base pairs (if the nucleic acid is double-stranded) or bases (if the nucleic acid is single stranded). As is described more fully below, single-stranded DNA primers, suitable for use in a polymerase chain reaction, are particularly preferred.

The nucleic acid for use in the methods of the invention is a nucleic acid capable of hybridising to the GNMT mRNA. Fragments of the GNMT gene and cDNAs derivable from the mRNA encoded by the GNMT gene are also preferred nucleic acids for use in the methods of the invention.

It is particularly preferred if the nucleic acid for use in the methods of the invention is an oligonucleotide primer which can be used to amplify a portion of the GNMT nucleic acid, particularly GNMT mRNA.

It is preferred if the nucleic acid is derived from a sample of the tissue in which prostate cancer is suspected or in which prostate cancer may be or has been found. Samples of prostate for example, may be obtained by surgical excision, laproscopy and biopsy, endoscopy and biopsy, and image-guided biopsy. The image for use in obtaining samples using image-guided biopsies of prostate tissue may be generated by ultrasound or by technetium-99-labelled antibodies or antibody fragments which bind or locate selectively at the prostate.

Although any sample containing nucleic acid derived from the patient is useful in the methods of the invention, it is preferred if the sample is selected from the group consisting of prostate tissue, blood, urine or semen. Prostate tissue can be obtained from a patient using standard surgical techniques. Cells derived from the prostate are found in small numbers in the urine and in the blood. If necessary these cells can be enriched from the patient sample, as discussed above. Although it is preferred that the sample containing nucleic acid from the patient is, or is derived directly from, a cell of the patient, such as a prostate cell, a sample indirectly derived from a patient, such as a cell grown in culture, is also included within the invention. Equally, although the nucleic acid derived from the patient may have been physically within the patient, it may alternatively have been copied from nucleic acid which was physically within the patient. The tumour tissue may be taken from the primary tumour or from metastases, and particularly may be taken from the margins of the tumour.

A second aspect of the invention provides a method for assessing a prostate cancer (including metastatic prostate cancer) and/or benign prostate hyperplasia treatment regime, the method comprising the step of determining the level of GNMT nucleic acid and/or protein in a sample from patients receiving the treatment regime. The sample type is typically of the type discussed above in relation to the first aspect of the invention, for example, a urine sample from the patient. The method may, for example, be used to provide information on the likelihood of the development of complications of prostate cancer in the patient. Thus, levels of GNMT may be used as surrogate markers in clinical trials of proposed treatments for prostate cancer. Measurement of GNMT may provide an overall assessment of how various factors affect the treatment of and progression of prostate cancer.

A third aspect of the invention provides a method for identifying a compound useful in modulating prostate function, for example in treating or preventing prostate cancer (including metastatic prostate cancer) and/or benign prostate hyperplasia, the method comprising the steps of a) determining whether a test compound is capable of suppressing production of, or activity of, GNMT in prostate tissue or a sample from a patient with, for example, prostate cancer and b) selecting a compound which is capable of suppressing production of, or activity of, GNMT in prostate tissue or a sample from a patient with, for example, prostate cancer. Other organ tissues may be more accessible for testing than the prostate. For example cells, which may be cancer cells, shed in the urine of the patient may be used. The compound may be administered to the patient or may be applied in vitro to the cells.

The method may comprise the step of determining whether a test compound is capable of suppressing production of, or activity of, GNMT in a sample, for example a urine sample from a patient, as discussed above.

In a fourth aspect the current invention provides a compound that targets Glycine N-methyltransferase (GNMT) protein and/or nucleic acid for use in treating prostate cancer.

In a fifth aspect the current invention provides the use of a compound that targets GNMT protein and/or nucleic acid in the manufacture of a medicament for treating prostate cancer.

In a sixth aspect the current invention provides a method for treating prostate cancer in a patient, comprising the step of administering to the patient an effective amount of a compound that targets GNMT protein and/or nucleic acid.

It is envisaged that the patient to be treated by the compounds, uses or methods of the current invention may be selected as a suitable candidate on the basis of screening for certain clinical markers for prostate cancer. Such markers may include, for example, Prostate specific antigen (PSA) levels, s-adenosyl-methionine (SAM) levels, s-adenosyl-homocysteine (SAH) levels, sarcosine levels, methionine levels, GNMT levels or any other suitable prostate cancer marker that would be evident to a person skilled in the art. Alternatively, the suitability of the patient for treatment with the compounds, uses and methods of the current invention may be determined by biopsy of the prostate cancer to be treated. Factors that may be assessed in such a biopsy may be global DNA methylation (or hypomethylation), chromosome stability, changes in expression of chromatin proteins, post-translational histone modification or other events that may indicate the condition of the prostate cancer. Such factors may be indications of the metastatic potential of the prostate cancer.

In an embodiment of any of the preceding aspects the compound reverses DNA hypomethylation. Not wishing to be bound by any particular theory, it is postulated that specific inhibition of GNMT by the compounds of the invention may lead to a reduction in the concentration of SAH in cells. SAH is a by-product of GNMT activity, as discussed above. While GNMT is resistant to the inhibitory effects of SAH (normally acts in a negative feedback loop with methyltransferases) other methyltransferases are not. Thus inhibition of GNMT may reduce SAH concentration while increasing overall methyltransferase activity in the cell. This may lead to a reversal, or at least reduction, in DNA hypomethylation. DNA hypomethylation in cancers other than prostate cancer has been shown to contribute to chromosomal instability and may help in the adaptive response of tumour cells to their micro-environment (see Example 1). Adaptation to different micro-environments may aid in tumour cell invasion and metastasis. It is envisaged that the property of preventing or reversing DNA hypomethylation with a compound of the invention may have the effect of reducing prostate cancer cell invasion and metastasis. Additionally, a reduction in DNA hypomethylation may aid in the restoration of the normal methylation patterns in prostate cancer cells, which may reduce prostate cancer cell growth. Protein methylation would also be expected to rise in cells where global methyltransferase activity increases. This may be used as a marker for increased methylation (methyltransferase activity) in the cell. For example, it is envisaged that levels of protein methylation may be determined by investigating methylation of, for example, histones at particular residues, for example, arginine or lysine residues. This may be achieved using commercially available antibodies. Such experiments may also be performed on a global scale to allow immunohistochemical detection of global methylation at a specific lysine or arginine residue in histones as would be understood by a person of skill in the art.

In an embodiment of any one of the preceding aspects the compound is an inhibitor of GNMT protein. The compound may be a small chemical entity. Such a compound may be identified by screening of libraries of compounds for the desired activity or may be designed rationally using structural information on GNMT or related proteins, which is available in the art. Methods and protocols for identifying such compounds are well known to those skilled in the art.

in an embodiment the compound may be selected from, but not limited to, 5-methyltetrahydrofolate pentaglutamate, sinefungin, 5-aza-cytidine, benzo-(a)-pyrene (Chen et al (2004) Cancer Res 64: 3617-3623) and any derivative or form thereof. These compounds may be used unmodified or alternatively may be used as lead compounds for the development of drug-like compounds. The compound may be 5-methyltetrahydrofolate pentaglutamate.

In a further embodiment, the compound may be selected from a compound exemplified in FIG. 5. For example, the compound may JR170, JR166, JR209, JR210 or JR276. In a preferred embodiment, the compound is JR170. It is envisaged that any one of compounds JR170, JR166, JR209, JR210 or JR276, and in particular JR170, may be used as lead compounds for the development of drug-like compounds.

The inhibitor of GNMT protein may alternatively be an antibody, antibody fragment or derivative thereof. Such an antibody may be raised by inoculation of a mammal (such as mouse, goat, sheep or horse) with purified GNMT, purified recombinant GNMT or fragment thereof. Alternatively such antibodies may be raised to artificially synthesised short polypeptides corresponding to specific regions of the GNMT amino acid sequence. Such antibodies may also be used in the detection methods for GNMT in samples, described above.

In a preferred embodiment, it is envisaged that the compound of any aspect of the current invention does not inhibit SAH sensitive methyltransferases. Therefore it is preferred that the compound is specific for inhibition of GNMT.

In a further aspect, the current invention provides a pharmaceutical formulation or kit of parts comprising a compound that targets GNMT protein, a means to target said compound to prostate cancer cells and a pharmaceutically acceptable carrier. Such pharmaceutical formulation or kit of parts may be for use in treating prostate cancer. The medicament or compound of any aspect of the current invention may be in a form suitable for oral delivery, intravenous delivery, intra-urethral delivery or delivery by any other suitable means in a pharmaceutically acceptable carrier. It is particularly preferred that the medicament or compound is in a form suitable for oral delivery.

In an alternative embodiment the compound of the current invention targets GNMT nucleic acid. Such a compound may be a small interfering RNA (siRNA) molecule. The siRNA may be exogenously expressed in the target prostate cells. Alternatively, the siRNA may be artificially synthesised and delivered in a suitable vector. This will depend on the bioavailability of compounds produced via each method in the target prostate cancer cells. The vector that may be used to deliver the siRNA molecule may be a liposome, a lipid micelle, a viral particle, or other suitable vector.

The current invention also provides a pharmaceutical formulation or kit of parts comprising an RNAi molecule or anti-sense oligonucleotide directed to GNMT, a means to target said RNAi molecule or anti-sense oligonucleotide to prostate cancer cells and a pharmaceutically acceptable carrier. Such a pharmaceutical formulation or kit of parts may be for use in treating prostate cancer.

In a further aspect, the current invention provides a screening method for selecting a drug-like compound or lead compound for the development of a drug-like compound considered to be useful in treating prostate cancer, comprising the steps of; a) determining the ability of a test compound to reduce GNMT activity; and b) selecting a compound that reduces GNMT activity.

An in vitro model may be most appropriate for performing the methods of the current invention. Thus, it may be appropriate to test compounds for an effect on production or activity of GNMT in an in vitro model system, for example in which the compound is applied in vitro to the cells.

Examples of appropriate in vitro models are:

(i) Primary culture of prostate cells/tissue from a patient with prostate cancer;
(ii) Human prostate cell lines;
(iii) Primary culture human cells from an uninvolved part of the prostate removed from patients with prostate cancer.

The test compound may be a small molecule, polypeptide or genetic construct, as will be well known to those skilled in the art. Compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

In an embodiment, the screening method may further comprise the steps of; a) determining the ability of the test compound to reduce SAH sensitive methyltransferase activity; and b) selecting a compound that does not reduce SAH sensitive methyltransferase activity. It is envisaged that such a test may be useful in determining the specificity of a test compound for GNMT inhibition.

It is intended that the compound that is identified by the screening methods of the preceding aspect of the invention is selected if it has an IC₅₀ value for GNMT of less than 1 mM, less than 1 μM or more preferably less than 1 nM.

It is envisaged that in all embodiments of the current invention, the test compound may be screened for the ability to inhibit SAH sensitive methyltransferase activity. This will provide some indication as to the specificity of the compound for GNMT. It is preferable that the compound is specific for GNMT inhibition in order to minimise potential side effects of therapy and in order to reduce concentrations of compound that may be required to bring about a therapeutic effect.

In an embodiment of the screening method of the current invention the effect of the test compound on the methylation of DNA is assessed. In a further embodiment the effect of the test compound on the methylation of protein is assessed. It is envisaged that histone protein methylation may be affected by GNMT activity and hence could be tested in cells.

In a yet further embodiment the effect of the test compound on levels of free methionine and/or SAM in cells may be assessed.

In a further embodiment of the screening method the effect of the test compound on the tissue levels of SAH, methionine and/or SAM is assessed.

In a yet further embodiment of the screening method the effect of the test compound on the ratio of SAM to SAH and/or sarcosine is assessed. It is envisaged that inhibition of GNMT may increase the ratio of SAM to SAH in cells or tissues where GNMT is expressed.

It is envisaged that in the screening method of the preceding aspect the test compound may be exposed to purified GNMT, or purified recombinant GNMT. This step will enable the GNMT inhibitory properties of the test compound to be assessed in further detail and for kinetic and biochemical data to be collected. Initial screens of test compounds may alternatively be performed on cell lysates containing either native GNMT or recombinant forms of GNMT, techniques that are well known to those skilled in the art.

In a further embodiment of the screening method of the current invention the test compound may be exposed to a cell expressing GNMT, or an extract of such a cell. This will enable assessment of the activity of the test compound on GNMT in a cellular environment and may provide an indication of the likely in vivo activity of the test compound, notwithstanding bioavailability and pharmacokinetics. It is envisaged that exposure of the cell or cell extract to androgen during or before assaying the test compound may aid in the simulation of the conditions found in a prostate cancer cell. Thus in an embodiment of the preceding aspect the cell or cell extract has been exposed to androgens such as testosterone, R1881, mibolerone and dihydrotestosterone. A further embodiment may comprise the step of assessing the effect of the test compound in combination with an androgen. Testing the test compound in the presence of androgen may provide an indication of the ability of the test compound to reduce prostate cancer cell growth and/or protect against the effect of androgens on the proliferation of prostate cancer cells. In the previous embodiment the androgen may be selected from, but not limited to, the group comprising testosterone, R1881, mibolerone or dihydrotestosterone. In a preferred embodiment the androgen is testosterone or dihydrotestosterone (DHT). In a further embodiment of the preceding aspect the cell or cell extract has been exposed to anti-androgens during or before assaying the test compound. This may be done in combination with androgens of the previous embodiment. Anti-androgens that may be used in this embodiment may be selected from, but not limited to, bicalutamide, cyproterone acetate and/or flutamide. Alternatively, other androgens or anti-androgens may be substituted in the preceding embodiments, as would be understood by a person of skill in the art.

In a further embodiment, the screening method may further comprise the step of determining the effect of the test compound on activation of androgen receptors in a cell or cell extract. Thus the activation of androgen receptors may be assayed in cells exposed to androgens and/or anti-androgens before or in conjunction with exposure to the test compound. It is intended that the androgens and/or anti-androgens of the preceding embodiments may be used in this embodiment.

The screening method of the preceding aspect may further comprise the step of determining the effect of the test compound on sarcosine, methionine, SAM and/or SAH levels in prostate cancer cells. It is envisaged that inhibition of GNMT by the test compound would have a measurable effect on the levels of these markers of GNMT activity in cells. It is envisaged that the cells will be lysed before testing the levels of sarcosine, methionine, SAM and/or SAH. However, it may also be useful to test the levels of these compounds, particularly sarcosine, secreted from the cells by testing the medium surrounding the cells.

It is envisaged that in the screening method of the preceding aspect the assay may be performed over a timescale of less than 72 hours, preferably less than 48 hours and more preferably over 24 hours or less.

The screening method of the current invention also comprises the step of preparing a pharmaceutical formulation comprising the selected compound. Said pharmaceutical formulation may be for use in treating prostate cancer.

In a further aspect, the current invention provides a method for aiding in the diagnosis of prostate cancer in a patient comprising obtaining a sample from the patient and assessing said sample for a marker of GNMT activity. By ‘a marker of GNMT activity’ is meant any by-product of GNMT activity that may be used to assess the presence of an active GNMT protein or fragment thereof. Specific markers of GNMT activity may include compounds selected from, but not limited to, the following: levels of s-adenosyl-methionine (SAM), s-adenosyl-homocysteine (SAH), sarcosine, methionine and/or GNMT. In the preceding aspect it is intended that the sample is a whole blood sample, a plasma sample, a serum sample, a urine sample, a prostate massage urine sample or a sample of seminal fluid. Alternatively, the sample may be a biopsy of cancerous or potentially cancerous tissue or surrounding tissue. It is envisaged that such a sample would be screened using techniques known in the art, such as Enzyme Linked Immuno-Sorbent Assay (ELISA) or High-Performance Liquid Chromatography (HPLC), as will be known to a person of skill in the art. It is further envisaged that levels of sarcosine, SAM and SAH may be determined using mass spectroscopy and/or nuclear magnetic resonance (NMR) methodologies well known to those skilled in the art. Assays of markers of GNMT activity may be conducted on lysed cells from a patient sample. It is intended that such techniques may be used in all aspects of the current invention where they may be appropriate.

In a further aspect the current invention provides s-adenosyl-methionine (SAM), s-adenosyl-homocysteine (SAH), sarcosine, methionine or GNMT for use as a biomarker for prostate cancer in a patient. These compounds may be used as biomarkers for diagnosis of prostate cancer, biomarkers for measuring the response to treatment for prostate cancer and/or for measuring the progression of prostate cancer in a patient. The levels of PSA may be measured in combination with markers of GNMT activity for the assessment of prostate cancer in a patient.

The current invention also provides a kit of parts comprising; a prostate biopsy or a sample from a patient; a reagent for detecting a marker of GNMT activity and a reagent for identifying or isolating prostate cells. In said kit the marker of GNMT activity is selected from, but not limited to, levels of s-adenosyl-methionine (SAM), s-adenosyl-homocysteine (SAH), sarcosine, methionine and/or GNMT. The sample may be a whole blood sample, a plasma sample, a serum sample, a urine sample, a prostate massage urine sample or a sample of seminal fluid. In a further embodiment the invention provides for use of said kit in a method for aiding in the diagnosis of prostate cancer in a patient.

It is intended that the patient be a mammalian patient. It is preferred that said mammalian patient be a human, but said patient can also be any one of, but not limited to, a dog, cat, horse, cow, rat, mouse, ape or monkey.

All documents referred to herein are incorporated herein, in their entirety, by reference.

The listing or discussion of a prior-published document in this specification should not necessarily be taken as an acknowledgment that the document is part of the state of the art or is common general knowledge.

The invention is now described in more detail by reference to the following, non-limiting, Figures and Examples.

### FIGURES

FIG. 1. Characterisation of androgen responses in LNCaP cells.

A) LNCaP cell growth is stimulated by the synthetic androgen R1881 (1 nM), as measured using the Sulphorhodamine B cell staining assay.

B) Immunoblotting of whole cell lysates following androgen treatment in LNCaP cells. Cells were grown in androgen-depleted medium for 72 hours, prior to the addition of R1881 for 36 hours. Results of lysates prepared from duplicate cultures for each treatment are shown for the androgen-regulated genes PSA and DRG1.

C) Secreted PSA levels were measured for culture medium from three replicate cultures grown as in part B, using the PSA radioimmunoassay.

D) RT-PCR analysis of PSA and DRG1 in RNAs prepared from LNCaP cells treated with R1881 over the time-course shown. From this analysis and subsequent real-time RT-PCR, it was concluded that 24 hours treatment with R1881 is optimal for microarray analysis of androgen responses.

E) Cluster analysis of genes demonstrating androgen-regulation by 2-fold or greater in LNCaP cells following 24 hour treatment with R1881 is shown. Microarray analysis was carried out on RNAs prepared from three bioreplicate cultures for the no ligand and R1881 treatments.

F) The ten genes demonstrating the most significant androgen regulation, identified from the microarray analysis are listed, together with the fold increase in expression, in the presence of R1881, compared to no ligand, as measured from the microarray analysis.

G) Semi-quantitative RT-PCR analysis of the genes identified in E and listed in F, for androgen regulation following 24 hour treatment with R1881.

H) Time course of GNMT expression, performed as in part F.

I) Immunoblotting of LNCaP cell lysates for GNMT, as described for part B.

J) The GNMT product S-adenosylhomocysteine (SAH) potentiates the androgen-stimulated growth of LNCaP cells. LNCaP cells were cultured in the absence or presence of 1 nM R1881 and increasing concentrations of SAH for 6 days at which point cell numbers were measured.

FIG. 2. Generation of a Tetracycline-Off regulatable LNCaP cell line

Thirteen LNCaP Tet-off clones were initially generated by retroviral transduction with the VP16-Tet-Off repressor (Clontech). Tet-Off regulation in these was assayed following transduction with the RevTRE-Luc Tet-Off-regulated reporter retrovirus (Clontech).

A) After 24 hours, media was changed and Doxtetracycline (Dox; 1 mg/ml) added as appropriate. Luciferase assays were performed after a further 72 hours of incubation. LNCaP was used as a negative control, and MCF-7 Tet Off and CHO-AA8-Luc Tet Off cells as positive controls. This analysis identified clone 3 as demonstrating the most significant Tet-Off regulation, with a low background. This line was, therefore, chosen for further study and named ‘LNCaP Tet Off’. The androgen regulation of PSA in LNCaP (B) and LNCaP Tet Off (C) and androgen regulated growth of LNCaP (D) and LNCaP Tet Off (E) cells were compared in a time course of androgen treatment. Cells were plated onto 6-well culture plates at 50,000 cells/well and treated in androgen-depleted media for 72 hours prior to the addition of R1881 (10 nM) and Dox (1 mg/ml), as appropriate. Media was removed after days 2, 4, 6 and 8 and PSA levels determined by radioimmunoassay. These experiments were performed independently three times. The mean values of the PSA measurements and growth are shown (with standard error bars). As seen in B-E, the LNCaP Tet Off line exhibits similar responses to androgen as LNCaP cells.

FIG. 3. in vitro assay of GNMT inhibitors. Assays of in vitro GNMT activity in the presence of compounds JR170, JR166, JR209, JR210 and JR276.

FIG. 4. Titration of inhibition of GNMT with JR170.

FIG. 5. Chemical structures of S-adenosyl-L-methionine (SAM), JR166, JR170, JR209, JR210 and JR276.

### EXAMPLE 1

### GNMT is Up-Regulated in Prostate Cancer Cells in Response to Androgen Stimulation

### Androgen Regulated Gene Responses in the LNCaP Prostate Cancer Cell Line

Gene profiling permits the identification and characterisation of AR-regulated genes, with the potential for the identification of novel prognostic markers, and therapeutic targets for the development of new treatments for prostate cancer.

In order to identify androgen-regulated genes in LNCaP cells, we have performed gene expression microarray profiling using the Applied Biosystems Genome Survey Microarray V2.0. Analysis of the data shows that expression of 318 genes is stimulated by greater than 2-fold within 24 hours of androgen treatment, whilst the expression of 272 genes is repressed by the same margin. These genes include known androgen-responsive genes, including PSA (KLK3), KLK2, DRG-1 and TMPRSS2.

Androgen action is mediated by the androgen receptor (AR), a transcription factor of the Nuclear Receptor superfamily. The androgen-bound AR stimulates prostate cancer growth through activation of a transcriptional programme which facilitates cancer cell proliferation and survival.

In order to identify androgen-responsive genes in prostate cancer cells, we have carried out gene expression microarray analysis. The LNCaP cell line was chosen for these studies, as this expresses AR, demonstrates androgen-regulated expression of androgen-responsive genes, such as the prostate cancer biomarker PSA, grows in an androgen-regulated manner in cell culture and forms androgen-dependent tumours in xenograft models (3).

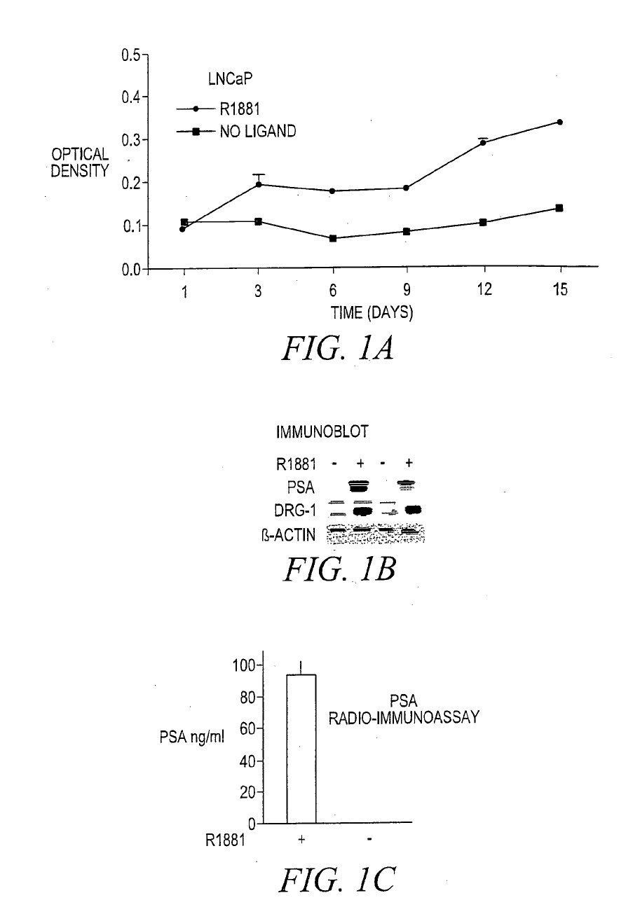

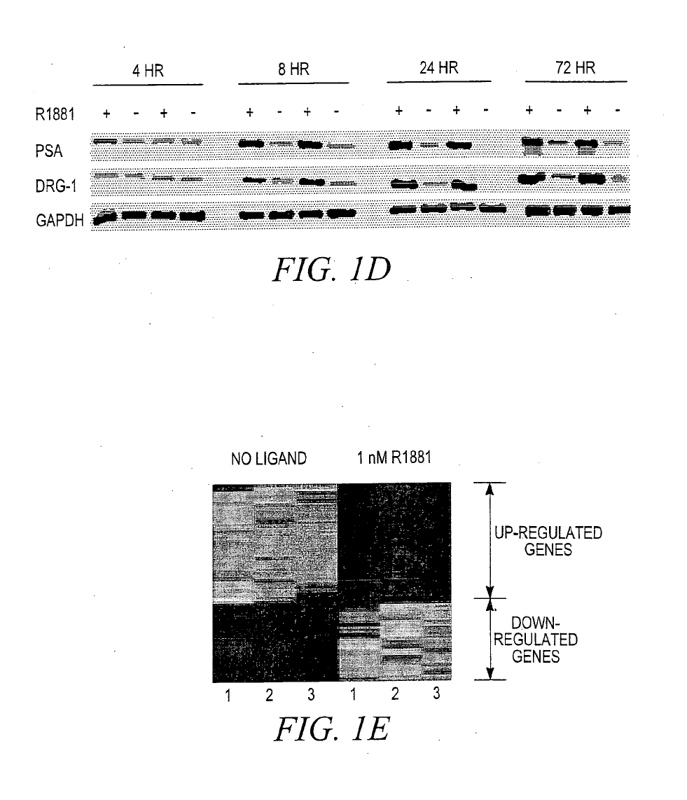

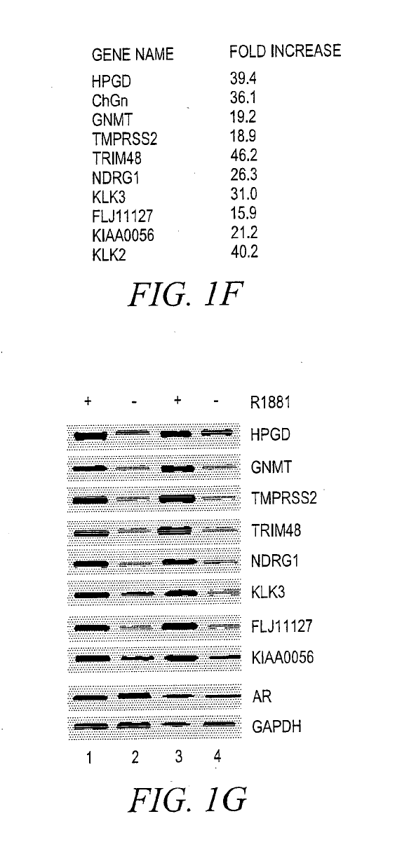

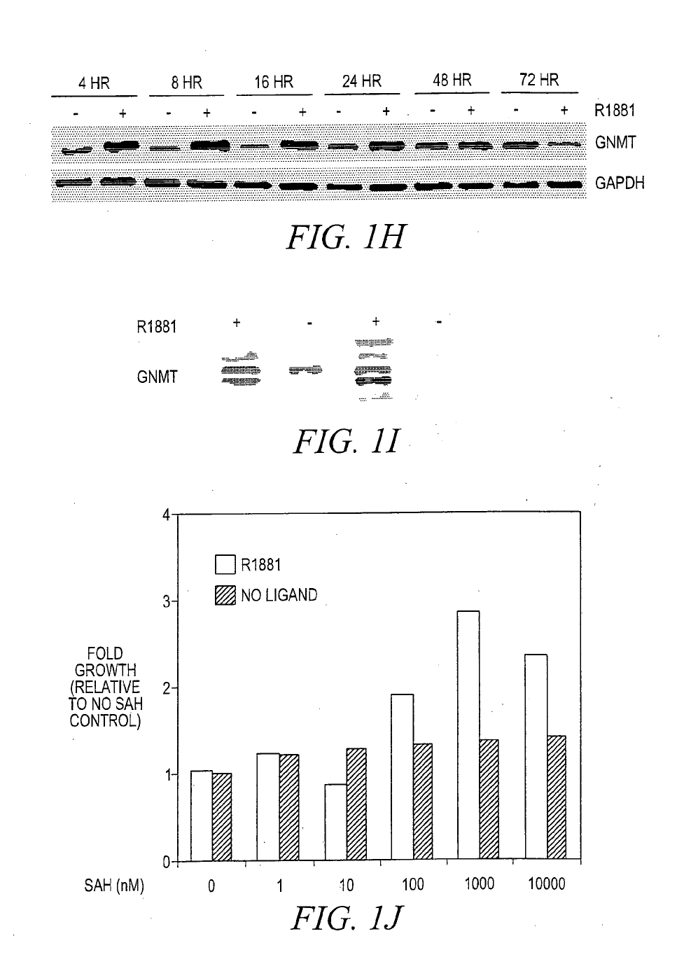

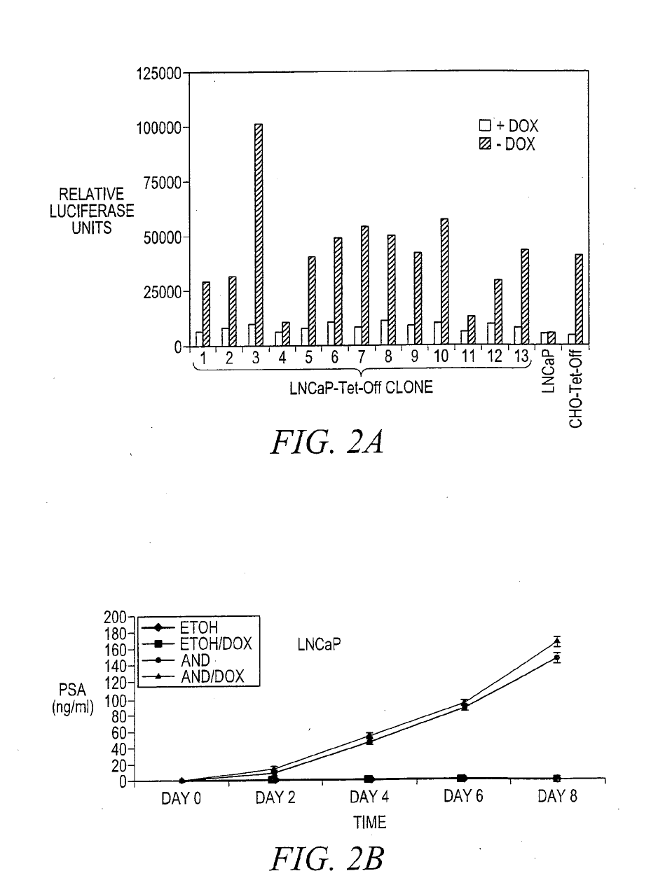

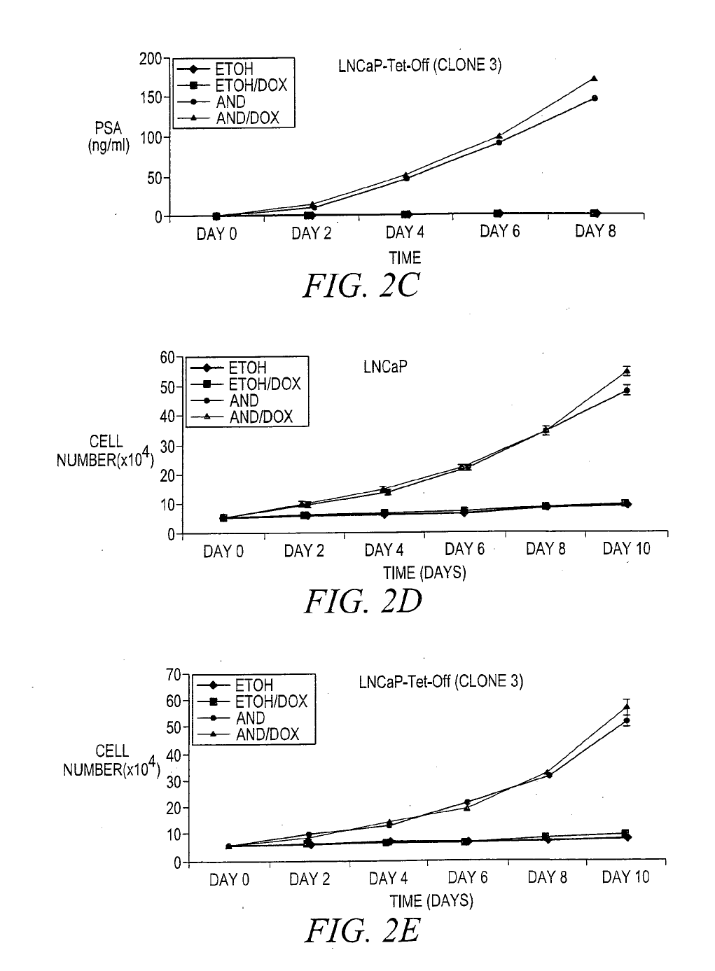

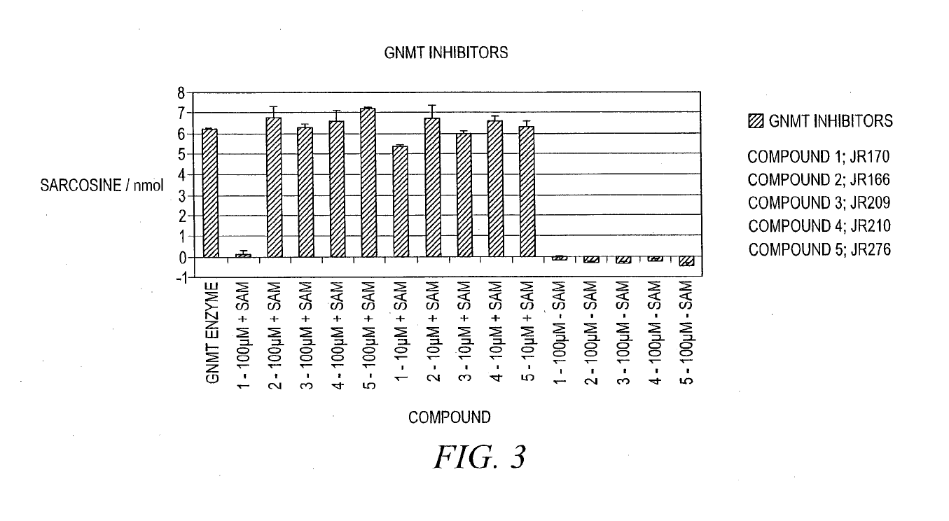

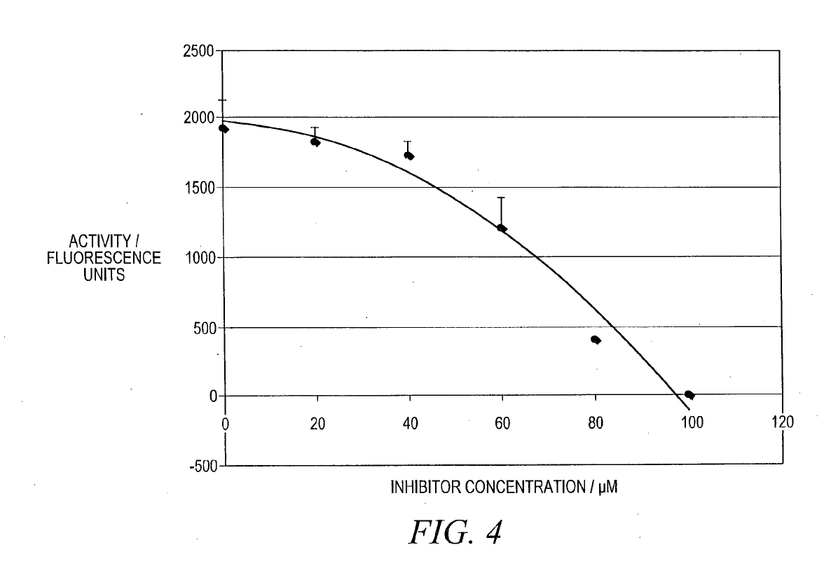

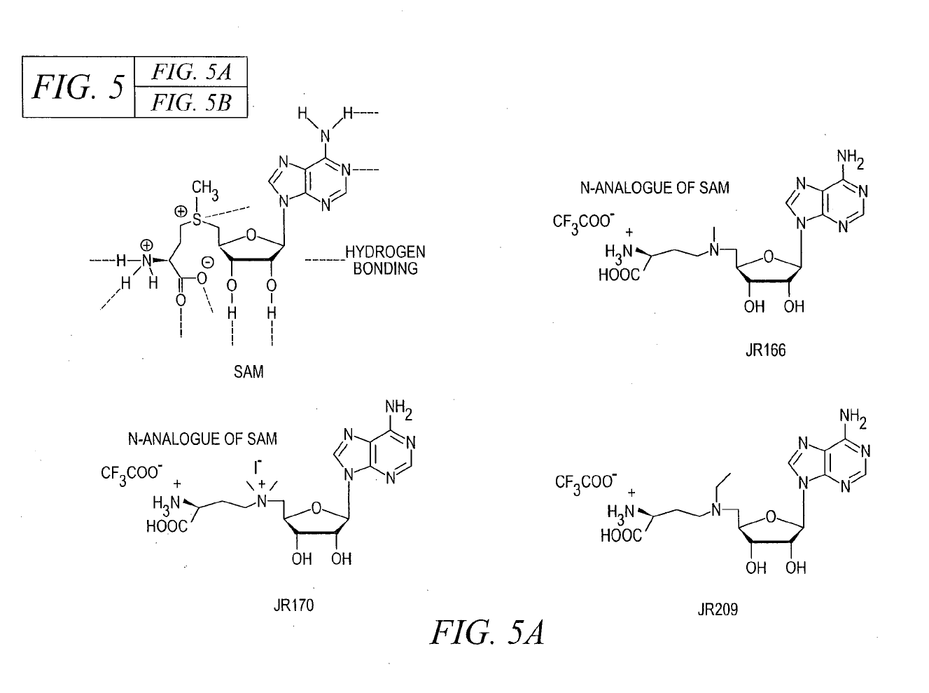

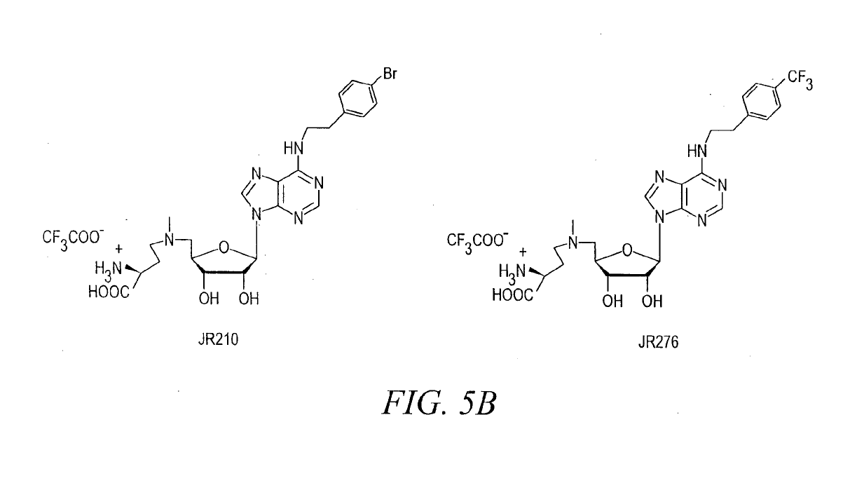

Detailed time course studies were undertaken to determine the time of androgen treatment for optimal expression of androgen-responsive genes (PSA and DRG-1) in the LNCaP cells (FIG. 1A-D). On the basis of these studies, RNA from five bio-replicate cultures were prepared from LNCaP cells treated with androgen for 24 hours, as well as the no ligand control. The RNAs were subjected to RNA quality assays and validated by real-time RT-PCR for two control genes (GAPDH and RPLPO) and the androgen regulated genes PSA, DRG-1 and GREB-1. These analyses showed that there was little variation between replicates and that the RNA's were of the highest quality. Three replicates from each treatment set were subsequently used for hybridisation to the Applied Biosystems (ABI) Human Genome Survey Microarray V2.0, which has probes for 29,098 genes, representing the most comprehensive coverage currently available for any human gene expression microarray platform. Raw data were quality assessed and filtered according to the recommendations supplied by the ABI1700 Data Analysis User Guide and the filtered data “vsn normalised” (4). Differential expression was assessed using liner models and empirical Bayes algorithms as described in (5). This analysis has robustly defined 456 probes whose expression was stimulated by androgen by two fold or greater, with positive lod scores (B values) for differential expression.

We also identified 349 probes whose expression was repressed by androgen by the same margins. In the case of the androgen up-regulated set, 140 probes show a four-fold or greater induction. Identified probes resulted in the lists of 318 up- and 272 down-regulated comprehensively annotated genes. Bonferroni corrected p-values of gene overrepresentation in functional categories, corresponding to the probabilities that genes in a particular category occur just by chance on the list, as determined by a reference list, were calculated, showing that in the up-regulated gene group Transferase, Isomerase, Dehydrogenase, Acetyltransferase, Oxidoreductase and Kinase functional groups are over-represented, if compared with the set of NCBI annotated genes. Further, the majority of already described androgen-regulated genes are present in the androgen-responsive gene set (e.g. ref. (6)), including PSA (KLK3; 30-fold up-regulated), KLK2 (32-fold), DRG-1 (24-fold) and TMPRSS2 (16-fold), and ten additional genes from the microarray analysis with significant fold inductions and, not previously identified as androgen regulated, have already been confirmed for androgen regulation by RT-PCR (FIG. 1G and data not shown).

We have ranked androgen-regulated genes according to the corresponding lod scores of their differential expression, as measured by our array analysis (FIGS. 1E and F) and have noted that the third most significant gene, showing a 16-fold increase in expression within 24 hours of androgen treatment, is the glycine N-methyltransferase (GNMT) gene (7). Further analysis has confirmed that GNMT gene expression is stimulated by androgen (FIG. 1G), with a time course showing potent induction within 4 hours of treatment (FIG. 1H). We have also confirmed that androgen treatment leads to induction of GNMT protein expression. These findings show for the first time that GNMT expression is potently stimulated by androgens. Importantly, previous studies have shown that GNMT expression demonstrates a very restricted tissue distribution, with most high-level expression being restricted to the liver, pancreas and prostate (7). Collectively, these data highlight GNMT as a potential new target molecule and marker for prostate cancer.

### GNMT as a Novel Androgen Regulated Gene for the Regulation of Methylation in Prostate Cancer

Biological methylation reactions which utilise S-adenosylmethionine (SAM) as a methyl donor encompass several key cellular and metabolic processes and include the methylation of phospholipids, proteins, DNA and RNA. This class of reaction is carried out by methyltransferases that are represented by a diverse group of distantly related proteins (8-10), with domains that catalyse the transfer of the methyl group from SAM to a molecular substrate, together with the in-situ generation of SAH. For most methyltransferases, SAH is known to have a higher affinity than SAM for the catalytic site, thereby acting as a potent feedback inhibitor of methyltransferase activity within the cell. For example, a decrease in the SAM/SAH ratio leading to an increase in SAH levels has been found to be associated with global DNA hypomethylation through inhibition of DNA methyltransferase activity (11). In most tissues, the balance between SAM and SAH and the subsequent regulation of global methylation activity resides in the ability to rapidly metabolise SAH through the enzyme S-adenosylhomocysteine hydrolase (SAH hydrolase). However, in a subset of tissues, it has been suggested that SAH levels can indeed be elevated at the expense of SAM, primarily through the activity of GNMT. GNMT is a phylogenetically conserved enzyme (12) that methylates Glycine to produce N-Methyl Glycine (sarcosine), which is known to be largely physiologically inert, and also generates SAH. However, unlike other methyltransferases, GNMT demonstrates a low affinity for SAH (13) and is, therefore, not subject to the same level of feedback inhibition imposed on other methyltransferases. Hence, for cells expressing GNMT, it has been proposed that the enzyme plays a pivotal role in regulating cellular methyltransferase activity, by decreasing levels of SAM and, perhaps more importantly, through the elevated production of the methyl transferase inhibitor SAH (14). Hence, it would be anticipated that GNMT could potentially lead to changes in global methylation, as part of the androgen response in prostate cancer. In order to address the potential consequence of this, we have examined LNCaP cell growth in the presence of androgen and SAH, at concentrations that overlap with the physiological levels found in serum (20-80 nM; (15)). As shown in FIG. 1J, addition of SAH to the culture medium clearly acts to potentiate the androgen-regulated growth of LNCaP cells, suggesting the possibility that GNMT expression in prostate cancer could contribute to androgen regulated tumour cell growth through the generation of SAH. Further, as demonstrated for several cell types, one consequence of increasing cellular SAH levels is to result in DNA hypomethylation (14). In prostate cancer, overall genome-wide DNA methylation is found to be significantly decreased in advanced and metastatic disease and it is, therefore, possible that androgen regulated GNMT expression could play a key role in mediating this. In other cancers, such DNA hypomethylation is associated with chromosomal instability and features in the adaptive response of the tumour to micro-environments, as seen in tumour invasion and metastasis. It has also recently been suggested that DNA hypomethylation in prostate cancer may well be important in the re-expression and maintenance of gene expression involved in acquiring and maintaining stem cell characteristics in the tumour cell population (reviewed in (16)).

### EXAMPLE 2

### Proposed Experiments to Further Analyse GNMT in Prostate Cancer

### Aims of the Proposed Work

Our recent, data have identified GNMT as a new and potentially important mediator of androgen regulated responses in prostate cancer cells, primarily acting through the regulation of global methylase activity in these cells, through the generation of SAH. These observations highlight new opportunities for exploiting GNMT in the context of prostate cancer in two ways. Firstly, GNMT may provide a useful new marker for prostate cancer, either as an androgen regulated protein in its own right or, perhaps more usefully, as an androgen-regulated enzyme whose activity may be easily measured (17). Secondly, GNMT may provide a new and valuable target for treatment, either through the development of specific, small molecule inhibitors (18), or by the utilisation of this androgen-induced enzyme activity in pro-drug anti-prostate tumour therapy. In order to evaluate GNMT in prostate cancer, we propose to further investigate the androgen regulation of GNMT expression in prostate cancer cells at the RNA and protein level, following treatment of cells with androgens and anti-androgens, and to define androgen regulation of the gene at the transcriptional level by generating reporter genes based on the GNMT promoter and using chromatin immunoprecipitation (ChIP) assays. GNMT expression in prostate cancer will be determined by immunohistochemical analysis of tissue microarrays, in order to define its relationship with prostate cancer progression. Finally, over expression and RNA interference studies will be carried out to investigate and further define the role of GNMT in prostate cancer cell growth and survival and, through the use of Low Density Array (LDA) microfluidic card analysis for the genes we have determined as showing androgen regulation, determine the role this enzyme may play in the epigenetic regulation of androgen regulated gene expression.

Collectively, these studies will further define the role of GNMT in the androgen response in prostate cancer and in the progression of the disease.

### Plan of Investigation:

a) Androgen regulation of GNMT: To date, we have evaluated the regulation of GNMT by the synthetic androgen R1881. We now propose to extend these initial studies by investigating how GNMT expression is regulated by the biological androgen Dihydro Testosterone (DHT) and various anti-androgens used in the treatment of prostate cancer, including biclutamide (casodex), cyproterone acetate (CPA) and flutamide. This will be carried out using real-time RT-PCR analysis (Applied Biosystems) and western blotting analysis using commercial GNMT antibodies (Abgent).
b) Androgen regulatory sequences in the GNMT gene: The GNMT gene encompasses 3.1 kb of DNA and is encoded on six exons located on Chromosome 6 (7). The transcriptional start and gene promoter are well mapped (19) and we have used this information to guide our analysis of the GNMT gene and flanking DNA. Our analysis of this region for sequences related to the canonical ARE (6) have identified two related motifs, with one being proximal to the gene promoter. We propose to further investigate these putative androgen response elements, by carrying out ChIP analysis of these regions from LNCaP cells. Briefly, this involves immunoprecipitation of sonicated, formaldehyde-fixed chromatin fragments prepared from LNCaP cells, with an antibody to AR, followed by PCR analysis of co-immunoprecipitated DNA for GNMT sequences encoding putative AREs. By comparing the amount of chromatin precipitated for each motif in the ChIP products from androgen treated and untreated LNCaP cells, it will be possible to identify those regions that preferentially bind AR in a ligand dependent manner.

In parallel studies, we will delineate regions mediating androgen responses in the GNMT gene, by carrying out a gene promoter analysis. Briefly, genomic DNA fragments encompassing the GNMT gene promoter, will be cloned upstream of a promoter-less firefly luciferase reporter gene. Initial round constructs will be transfected into LNCaP cells, together with a control luciferase (renilla) and reporter gene activity measured in the presence and absence of androgen. Genomic DNA fragments demonstrating androgen responsiveness will be further analysed by sequential deletion of DNA, to further delineate response elements. Responsive regions will be analysed in detail by ChIP, as outlined above. Finally, delineated regulatory sequences, together with mutant versions of these, will be re-cloned into a reporter vector with a basal promoter (pGL3Luc; Promega) to confirm the ability of these sequences to mediate androgen regulated gene expression.

c) Conditional overexpression and knockdown of GNMT in LNCaP cells: We propose to study the effect of GNMT on LNCaP cell growth and cell survival by making lines in which GNMT expression can be conditionally regulated. To enable this, we have made an LNCaP line in which transgene expression can be regulated in a “tetracycline-off” manner (FIG. 2). Using this line, we propose to firstly over-express GNMT in a conditional manner using the pREV-TRE tet-off regulated vector (see our ref. Buluwela et al. 2005). Similarly, we will conditionally express an optimised shRNA against GNMT in the tetracycline-off compatible, regulatable RNA interference vector SIN-TREmiR30-PIG (www.openbiosystems.com). To facilitate these studies, we have already obtained and characterised in transient transfection experiments a full coding region EST expression clone for GNMT (www.rzpd.de) and two shRNA expression constructs against GNMT (www.openbiosystems.com). The growth of cells over-expressing GNMT will be investigated in the presence and absence of tetracycline, both in the presence and absence of androgen. The effect of SAM, a key substrate for GNMT, on cell growth under these conditions will also be investigated. Similarly, experiments addressing the effect of GNMT knockdown in LNCaP will be carried out using the same culture treatments. Using both sets of lines, the effects of GNMT on cell cycle and survival will be further investigated by similar growth experiments, which will be analysed by FACS (see our ref. Pike et al 2004).
d) LDA analysis of GNMT target genes: As seen in FIG. 1J, SAH acts to potentiate androgen regulated growth. It is, therefore, possible that GNMT, through the generation of SAH, may act to modify the androgen response in prostate cancer cells, with such an effect being measurable through how the androgen gene response is affected. Our array studies have identified a panel of 590 androgen regulated genes that define the androgen response in LNCaP cells. We have already designed and started to use LDA microfluidic cards (Applied biosystems) representing all 318 androgen up-regulated (two fold or greater) and the top 55 androgen down-regulated genes in further studies of the androgen response. These cards generate real-time quantitative RT-PCR data for each gene represented on the array for one cDNA sample. We now propose to use the same LDA card format to address how GNMT may modify the androgen gene response, by comparing androgen regulated gene expression in LNCaP Tet-Off cells compared to the LNCaP Tet-Off lines either overexpressing GNMT, or demonstrating GNMT knockdown by RNAi. By doing this, we will be able to identify those genes whose expression is affected by GNMT, thereby defining how the enzyme may be involved in the androgen response. These genes would be subsequently investigated for evidence of epigenetic alteration through DNA methylation, as measured by methylation-specific PCR around the gene promoter and for methylation associated with the chromatin of these candidate genes, using ChIP analysis for methylated histones using commercially available antibodies to the various methylated isoforms.
e) GNMT expression in prostate tumours: We aim to evaluate the expression of GNMT in prostate tumours by immunohistochemistry using AccuMax prostate cancer tissue microarrays (TMA's; Stretton Scientific), which represent stage II (where the cancer involves more tissue within the prostate but is organ confined), Ill (where the cancer has spread outside the prostate to nearby tissue) and IV (where the cancer has metastasised to lymph node or other tissues) prostate cancer tissue samples with full clinical details. Each TMA represents 40 samples in 84 spots, including two pairs of spots from normal prostate, for comparison. We aim to probe such TMA's with two commercially available Rabbit polyclonal antisera raised against N-terminal and C-terminal human GNMT sequences (Abgent). In all, we aim to probe two arrays for each antibody, for each disease stage (80 cases) and score these for GNMT expression. The significance of this staining will be analysed using statistical support provided to us by Imperial College London, with a view to developing an understanding of the association of GNMT expression in prostate cancer disease and disease progression.

### REFERENCES

1. Carter, H. B., Coffey, D. S. (1990) The prostate: an increasing medical problem. Prostate, 16, 39-48.
2. McConnell, J. D. (1991) Physiologic basis of endocrine therapy for prostatic cancer. Urol Clin North Am, 18, 1-13.
3. Sobel, R. E. and Sadar, M. D. (2005) Cell lines used in prostate cancer research: a compendium of old and new lines—part 1. J Urol, 173, 342-359.
4. Huber, W., von Heydebreck, A., Sultmann, H., Poustka, A. and Vingron, M. (2002) Variance stabilization applied to microarray data calibration and to the quantification of differential expression. Bioinformatics, 18 Suppl 1, S96-104.
5. Smyth, G. K. (2004) Linear models and empirical bayes methods for assessing differential expression in microarray experiments. Stat Appl Genet Mol Biol, 3, Article 3.
6. Nelson, P. S., Clegg, N., Arnold, H., Ferguson, C., Bonham, M., White, J., Hood, L. and Lin, B. (2002) The program of androgen-responsive genes in neoplastic prostate epithelium. Proc Natl Acad Sci USA, 99, 11890-11895.
7. Chen, Y. M., Chen, L. Y., Wong, F. H., Lee, C. M., Chang, T. J. and Yang-Feng, T. L. (2000) Genomic structure, expression, and chromosomal localization of the human glycine N-methyltransferase gene. Genomics, 66, 43-47.
8. Krause, C. D., Yang, Z. H., Kim, Y. S., Lee, J. H., Cook, J. R. and Pestka, S. (2006) Protein arginine methyltransferases: Evolution and assessment of their pharmacological and therapeutic potential. Pharmacol Ther.
9. Jeltsch, A. (2006) Molecular enzymology of mammalian DNA methyltransferases. Curr Top Microbial Immunol, 301, 203-225.
10. Dillon, S. C., Zhang, X., Trievel, R. C. and Cheng, X. (2005) The SET-domain protein superfamily: protein lysine methyltransferases. Genome Biol, 6, 227.
11. Castro, R., Rivera, I., Martins, C., Struys, E. A., Jansen, E. E., Glade, N., Graca, L. M., Blom, H. J., Jakobs, C. and de Almeida, I. T. (2005) Intracellular S-adenosylhomocysteine increased levels are associated with DNA hypomethylation in HUVEC. J Mol Med, 83, 831-836.
12. Ogawa, H., Gomi, T. and Fujioka, M. (1993) Mammalian glycine N-methyltransferases. Comparative kinetic and structural properties of the enzymes from human, rat, rabbit and pig livers. Comp Biochem Physiol B, 106, 601-611.
13. Huang, Y., Komoto, J., Konishi, K., Takata, Y., Ogawa, H., Gomi, T., Fujioka, M. and Takusagawa, F. (2000) Mechanisms for auto-inhibition and forced product release in glycine N-methyltransferase: crystal structures of wild-type, mutant R175K and S-adenosylhomocysteine-bound R175K enzymes. J Mol Biol, 298, 149-162.
14. Ulrey, C. L., Liu, L., Andrews, L. G. and Tollefsbol, T. O. (2005) The impact of metabolism on DNA methylation. Hum Mol Genet, 14 Spec No 1, R139-147.
15. Kerins, D. M., Koury, M. J., Capdevila, A., Rana, S, and Wagner, C. (2001) Plasma 5-adenosylhomocysteine is a more sensitive indicator of cardiovascular disease than plasma homocysteine. Am J Olin Nutr, 74, 723-729.
16. Schulz, W. A. and Hatina, J. (2006) Epigenetics of prostate cancer: beyond DNA methylation. J Cell Mol Med, 10, 100-125.
17. Santos, F., Amorim, A. and Kompf, J. (1995) Specific staining of glycine N-methyltransferase. Electrophoresis, 16, 1898-1899.
18. Yeo, E. J., Briggs, W. T. and Wagner, C. (1999) Inhibition of glycine N-methyltransferase by 5-methyltetrahydrofolate pentaglutamate. J Biol Chem, 274, 37559-37564.
19. Tseng, T. L., Shih, Y. P., Huang, Y. C., Wang, C. K., Chen, P. H., Chang, J. G., Yeh, K. T., Chen, Y. M. and Buetow, K. H. (2003) Genotypic and phenotypic characterization of a putative tumor susceptibility gene, GNMT, in liver cancer. Cancer Res, 63, 647-654.
20. Luka et al. (2004) Proteins 57: 331-337.
21. Luka & Wagner (2003) BBRC 312: 1067-1072
22. Luka et al., (2006) Transgenic Res. 15: 393-397

### EXAMPLE 3

### Inhibition of GNMT by Putative Inhibitor Compounds

FIG. 3 shows the results of in vitro assays of GNMT activity in the presence of putative GNMT inhibitor compounds.

Human, recombinant GNMT was expressed in Escherichia coli and purified as described by Luka and Wagner (2003) “Expression and purification of glycine N-methyltransferases in Escherichia coli.” Protein expression and purification 28(2): 280-6. The recombinant GNMT was used to assay the activity of the compounds JR166, JR170, JR209, JR210 and JR276 as putative inhibitors of the glycine N-methyl transferase activity of GNMT. The chemical structures of the putative inhibitor compounds are shown in FIG. 5.

### Experimental Methods Used for GNMT Inhibition Assays

Enzyme reactions (200 μl) consisted of 0.2 M Tris buffer (pH 9.0), 8 mM glycine, 0.5 mM S-adenosyl-L-methionine (SAM), and 2 μg recombinant enzyme and were incubated at 25° C. for 30 minutes. Following incubation, assays were heat inactivated at 75° C. for 10 minutes and the amount of N-methyl glycine (sarcosine) measured using a flourimetric assay (BioVision, California, USA).

For inhibition studies, GNMT activity was measured in a standard enzyme reaction (column 1 of FIG. 3) in the presence of 100 μM (columns 2-6 of FIG. 3) and 10 μM (columns 7-11 of FIG. 3) of each of the compounds. Control reactions, using each compound at 100 μM without SAM produced no sarcosine (columns 12-16 of FIG. 3). The results show the average activity of three replicate assays, with error bars (standard error of the mean).

Only compound JR170 showed an inhibition of in vitro GNMT activity (columns 2 and 7 of FIG. 3).

### EXAMPLE 4

### Further Analysis of GNMT Inhibition by Compound JR170

FIG. 4 shows the results of further analysis of the in vitro inhibition of GNMT activity by compound JR170.

Human, recombinant GNMT was expressed in Escherichia coli and purified as described by Luka and Wagner (2003) supra. The recombinant GNMT was used to titrate the inhibitory activity of JR170 on the glycine N-methyl transferase activity of GNMT.

### Experimental Methods

Enzyme reactions (200 μl) consisted of 0.2 M Tris buffer (pH 9.0), 8 mM glycine, 0.5 mM S-adenosyl-L-methionine (SAM), and 2 μg recombinant enzyme and were incubated at 25° C. for 30 minutes. Following incubation, assays were heat inactivated at 75° C. for 10 minutes and the amount of N-methyl glycine (sarcosine) measured using a flourimetric assay (BioVision, California, USA). For titration studies, GNMT activity was measured in a standard enzyme reaction in the presence of a range of JR170 concentrations from 0-100 μM.

The results displayed in FIG. 4 show the average activity of three replicate assays, with error bars (standard error of the mean).

1. A method for aiding in the assessment of prostate cancer (including metastatic prostate cancer) and/or benign prostate hyperplasia in a patient, wherein the method comprises the step of determining the level of Glycine N-methyltransferase (GNMT) nucleic acid and/or protein in a sample from the patient.

2. The method of claim 1 wherein the patient is; (i) at risk of developing prostate cancer; or (ii) has already been diagnosed with prostate cancer; or (iii) has undergone treatment for prostate cancer.

3. The method of claim 1 wherein the sample is a blood sample, a urine sample, a prostate massage urine sample, a lymph sample or a sample of seminal fluid.

4. The method of claim 1, wherein the method is for assessing the likely progression of prostate cancer in the patient.

5. The method of claim 1 wherein the method is for assessing and/or predicting the development of prostate cancer.

6. The method of claim 1, wherein the method is for diagnosing prostate cancer in the patient.

7. The method of claim 1, wherein the method is for assessing the response of the patient to treatment.

8. The method of claim 7 wherein the method is for assessing the likely progression of response of the patient to treatment.

9. The method of claim 1, wherein the method is for choosing patients for treatment for prostate cancer.

10. The method of claim 1, wherein the method is for monitoring response of patients to treatment or for monitoring relapse in patients.

11. The method of claim 1, wherein the method is for assessing the progress of clinical trials.

12. A method for assessing a prostate cancer (including metastatic prostate cancer) and/or benign prostate hyperplasia treatment regime, the method comprising the step of determining the level of GNMT nucleic acid and/or protein in a sample from patients receiving the treatment regime.

13-35. (canceled)

36. A screening method for selecting a drug-like compound or lead compound for the development of a drug-like compound considered to be useful in treating prostate cancer, comprising the steps of;
determining the ability of a test compound to reduce GNMT activity; and
selecting a compound that reduces GNMT activity.

37. The screening method of claim 36 further comprising the steps of;
determining the ability of the test compound to reduce SAH sensitive methyltransferase activity; and
selecting a compound that does not reduce SAH sensitive methyltransferase activity.

38. The screening method of claim 36 wherein the compound is selected if it has an IC50 value for GNMT of less than 1 mM, less than 1 μM or less than 1 nM.

39. The screening method of claim 36 wherein the effect of the test compound on the methylation of DNA is assessed.

40. The screening method of claim 36 wherein the effect of the test compound on the methylation of protein is assessed.

41. The screening method of claim 36 wherein the effect of the test compound on levels of free methionine and/or SAM in cells is assessed.

42. The screening method of claim 36 wherein the effect of the test compound on the tissue levels of SAH, methionine and/or SAM is assessed.

43. The screening method of claim 36 wherein the effect of the test compound on the ratio of SAM to SAH and/or sarcosine is assessed.

44. The screening method of claim 36 wherein the test compound is exposed to purified GNMT, or purified recombinant GNMT.

45. The screening method of claim 36 wherein the test compound is exposed to a cell expressing GNMT, or an extract of such a cell.

46. The screening method of claim 45 wherein the cell or cell extract has been exposed to androgen.

47. The screening method of claim 46 wherein the cell or cell extract has been exposed to testosterone, R1881, dihydrotestosterone (DHT) or mibolerone.

48. The screening method of claim 36 further comprising the step of assessing the effect of the test compound in combination with an androgen.

49. The screening method of claim 48 wherein the androgen is selected from, but not limited to, the group comprising testosterone, R1881, mibolerone or dihydrotestosterone.

50. The screening method of claim 48 wherein the androgen is testosterone or DHT.

51. The screening method of claim 45 wherein the cell or cell extract has been exposed to anti-androgen.

52. The screening method of claim 51, wherein the anti-androgen is bicalutamide, cyproterone acetate or flutamide.

53. The screening method of claim 36 further comprising the step of determining the effect of the test compound on activation of androgen receptors in a cell or cell extract.

54. The screening method of claim 36 further comprising the step of determining the effect of the test compound on sarcosine, methionine, SAM and/or SAH levels in prostate cancer cells.

55. The screening method of claim 36 wherein the assay is performed over a timescale of less than 24 hours, preferably less than 72 hours, preferably less than 48 hours and more preferably over 24 hours or less.

56-64. (canceled)
